(19) United States
(12) Patent Application Publication
(10) Pub. No.: US 2023/0178179 A1
(43) Pub. Date: Jun. 8, 2023

(54) Memory-efficient whole genome assembly of long reads

(71) Applicants: Baris Ekim, Somerville, MA (US); Bonnie Berger Leighton, Newtonville, MA (US); Rayan Chikhi, Paris (FR)

(72) Inventors: Baris Ekim, Somerville, MA (US); Bonnie Berger Leighton, Newtonville, MA (US); Rayan Chikhi, Paris (FR)

(21) Appl. No.: 17/903,654

(22) Filed: Sep. 6, 2022

Related U.S. Application Data
(60) Provisional application No. 63/241,048, filed on Sep. 6, 2021.

(51) Int. Cl. G16B 30/00; G16B 45/00

(52) U.S. Cl. CPC G16B 30/00; G16B 45/00

(57) ABSTRACT

A method for computation- and memory-efficient DNA sequencing. In one embodiment, the approach herein is used to facilitate genome assembly for state-of-the-art and low-error long-read data. In this embodiment, the approach herein implements a minimizer-space de Bruijn graph, which—instead of building an assembly over sequence bases (in a base-space wherein an alphabet sequence comprises nucleotide letters)—performs assembly in a minimizer-space (wherein an alphabet sequence comprises an ordered sequence of minimizers), and later converts the assembly back to base-space assemblies. Specifically, and in a preferred implementation, each read is initially converted to an ordered sequence of its minimizers. The order of the minimizers is maintained to facilitate reconstructing the entire genome as an ordered list. To aid in assembly of higher-error rate data, a partial order alignment (POA) algorithm designed to operate in minimizer-space instead of base-space in implemented, and it effectively corrects only the bases corresponding to minimizers in the reads.

### STATEMENT OF FEDERALLY SPONSORED RESEARCH OR DEVELOPMENT

This invention was made with Government support under NIH R01HG010959 and NIH R35GM141861. The Government has certain rights in this invention.

### BACKGROUND

### Technical Field

This disclosure relates generally to computation- and memory-efficient long read DNA sequencing methods.

### Related Art

DNA sequencing data continues to improve from long reads of poor quality, used to assemble the first human genome, to Illumina short reads with low error rates (≤1%), and now to longer reads with low error rates. A tantalizing possibility is that DNA sequencing will eventually converge to long, nearly-perfect reads. To achieve this goal, new technologies will require algorithms that are both efficient and accurate for important sequence analysis tasks, such as genome assembly.

Efficient algorithms for sequence analysis have played a central role in the era of high-throughput DNA sequencing. Many analyses, such as read mapping, genome assembly, and taxonomic profiling, have benefited from milestone advances that effectively compress, or sketch, the data. These include fast full-text search with the Burrows-Wheeler transform, space-efficient graph representations with succinct de Bruijn graphs, and lightweight databases with MinHash sketches. Large-scale data re-analysis initiatives further incentivize the development of efficient algorithms, as they aim to re-analyze petabases of existing public data.

There has traditionally been a tradeoff between algorithmic efficiency and loss of information, however, at least during the initial sequence processing steps. Consider short-read genome assembly: the non-trivial insight of chopping up reads into k-mers, thereby bypassing the ordering of k-mers within each read, has unlocked fast and memory-efficient approaches using de Bruijn graphs; yet, the short k-mers—chosen for efficiency—lead to fragmented assemblies. In modern sequence similarity estimation and read mapping approaches, information loss is even more drastic, as large genomic windows are sketched down to comparatively tiny sets of minimizers, which index a sequence (window) by its lexicographically smallest k-mer, and enable efficient but sometimes inaccurate comparisons between gigabase-scale sets of sequences.

There remains a need to provide improved techniques for DNA sequencing that are memory-efficient.

### BRIEF SUMMARY

The subject matter hereof describes a method for a highly-efficient DNA sequence analysis technique. In one embodiment, the approach herein is used to facilitate genome assembly for state-of-the-art and low-error long-read data. In this embodiment, the approach herein implements a minimizer-space de Bruijn graph, which—instead of building an assembly over sequence bases (in a base-space wherein an alphabet sequence comprises nucleotide letters ACGT)—performs assembly in a minimizer-space (wherein an alphabet sequence comprises an ordered sequence of minimizers), and later converts the assembly back to base-space assemblies. Specifically, and in a preferred implementation, each read is initially converted to an ordered sequence of its minimizers. The order of the minimizers is maintained to facilitate reconstructing the entire genome as an ordered list. To aid in assembly of higher-error rate data, a partial order alignment (POA) algorithm designed to operate in minimizer-space instead of base-space in implemented, and it effectively corrects only the bases corresponding to minimizers in the reads.

The basic approach herein leverages the notion that minimizers can themselves make up atomic tokens of an extended alphabet, which enables efficient long-read assembly that, along with error correction, leads to preserved accuracy. By performing assembly using a minimizer-space de Bruijn graph (in the preferred embodiment), the approach herein reduces the amount of data input to the assembler, preserving accuracy, lowering running time, and decreasing memory usage (e.g., several orders of magnitude) compared to current assemblers.

The foregoing has outlined some of the more pertinent features of the subject matter. These features should be construed to be merely illustrative. Many other beneficial results can be attained by applying the disclosed subject matter in a different manner or by modifying the subject matter as will be described.

### BRIEF DESCRIPTION OF THE DRAWINGS

For a more complete understanding of the subject matter and the advantages thereof, reference is now made to the following descriptions taken in conjunction with the accompanying drawings, in which:

FIG. 1A depicts a comparison between a classical approach, and the minimizer-space de Bruijn graph (mdBG) of this disclosure;

FIG. 1B depicts of an overview of a representative assembly pipeline using a minimizer-space de Bruijn graph according to the approach herein;

FIG. 1C depicts an overview of a minimizer-space partial order alignment (POA) process that is utilized to mitigate sequencing errors according to a preferred embodiment;

FIG. 2 depicts an bucketing algorithm of ordered lists of minimizers to facilitate the minimizer-space POA procedure;

FIG. 3 depicts a neighbor collection algorithm for the minimizer-space POA procedure;

FIG. 4 depicts a minimizer-space graph construction and consensus generation algorithm for the minimizer-space POA procedure;

FIG. 5 depicts Table 1, showing assembly statistics of D. melanogaster real HiFi reads, simulated perfect reads, and Human real HiFi reads;

FIG. 6 is a graph depicting the effect of minimizer-space POA correction on mdBG assembly in an example embodiment;

FIG. 7 depicts graphs of robustness of assemblies;

FIG. 8 depicts a pangenome mdBG of a set of bacterial genomes and associated retrieval of anti-microbial resistance genes;

FIG. 9 depicts Table 2, showing metagenome assembly statistics of the Zymo D6331 dataset (left) and the ATCC MSA-1003 dataset (right) using a known approach, and the approach of this disclosure; and

FIG. 10 depicts Table 3, showing assembly statistics using both universe minimizers and universe minimizers with Locally Consistent Parsing (LCP) of D. melanogaster real HiFi reads, simulated perfect reads, and Human real HiFi reads.

### DETAILED DESCRIPTION

Genome assembly is the computational task of assembling (stitching together) sequencing reads into a single genomic sequence per chromosome. The prevailing approach, de novo assembly, is naively resource-intensive because it requires pairwise comparisons between all possible pairs of reads. The following describes a method for genome assembly using minimizer-space de Bruijn graphs according to a preferred embodiment of this disclosure. As will be seen, the techniques of this disclosure leverage an insight of language models, namely, that words (or sentence fragments), instead of letters, can be used as tokens (small building blocks) in a computational model of a natural language. Likewise, and according to this disclosure, sequence analysis is carried out using a data structure referred to as a minimizer-space de Bruijn graph (mdBG) where, instead of single nucleotides as tokens of the de Bruijn graph, short sequences of nucleotides known as minimizers, which allow for an even more compact representation of the genome in minimizer space, are utilized. As will be seen, minimizer-space de Bruijn graphs store only a small fraction of the nucleotides from the input data while preserving the overall graph structure, enabling these graphs to be orders of magnitude more efficient than classical de Bruijn graphs. By doing so, an analysis tool that implements the approach herein can reconstruct whole genomes from accurate long-read data in much shorter time frames, while using significantly less memory and still achieving similar accuracy.

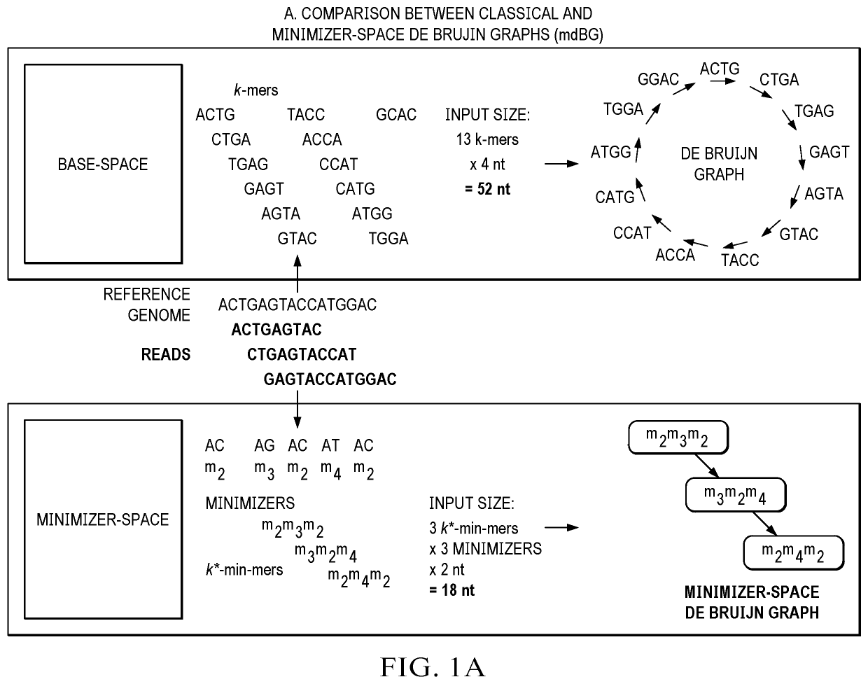

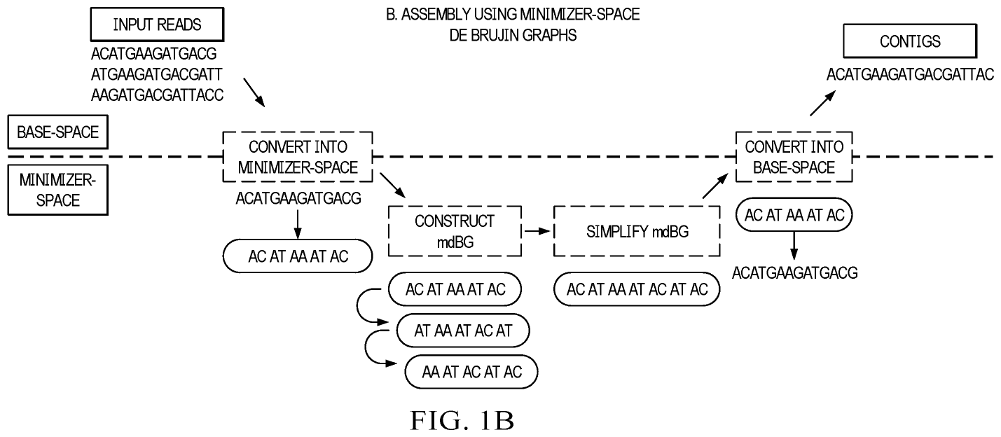

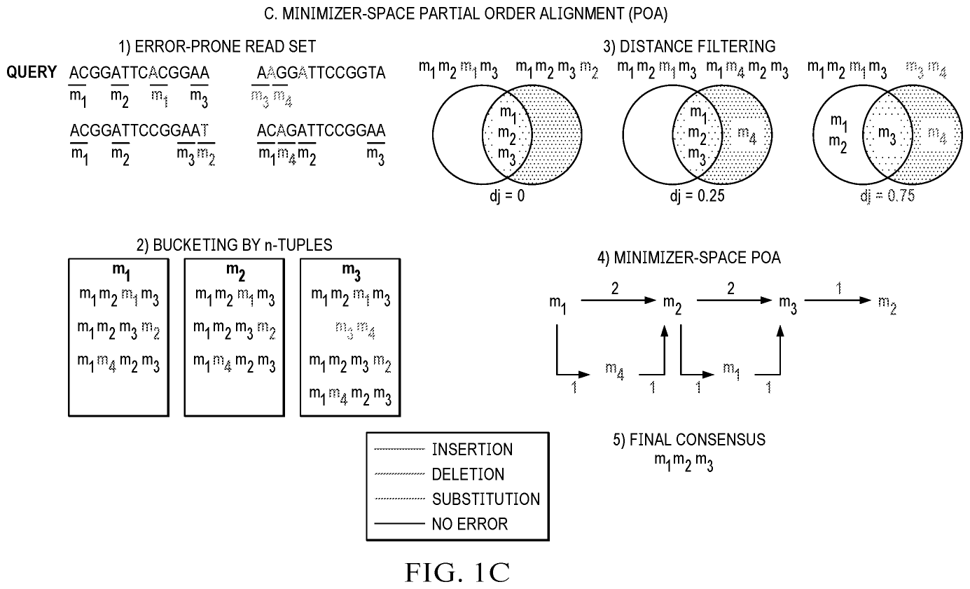

FIG. 1A depicts the basic approach and, in particular, the comparison between a known technique, and the minimizer-space de Bruijn graph of this disclosure. The top portion depicts the classical base-space technique (box 100) commonly used for genome assembly, and the bottom portion depicts the minimizer-space technique (box 102) of this disclosure. In this example, the center section 104 shows a toy reference genome, along with a collection of sequencing reads. The top box 100 shows k-mers (k=4) collected from the reads 101, which are the nodes of the classical de Bruijn graph 103. In this example, the input size of 52 nucleotides (nt) is depicted in boldface. The bottom box 102 shows the position of minimizers 105 in the reads for l=2, and any l-mer starting with nucleotide “A” is chosen as a minimizer. k′-min-mers (using notation k′=3 here to differentiate from classical k-mers) 107 are tuples of k′ minimizers as ordered in reads, which constitute the nodes of the minimizer-space de Bruijn graph 109. Creating k′-min-mers from the minimizer-space representation of reads allows for a reduction in input size, because the only bases stored in a k′-min-mer are the bases of the chosen minimizers. The reduced input size to 18 nucleotides (nt) is depicted in boldface. The minimizer-space representation accelerates the construction and traversal of the de Bruijn graph while reducing memory consumption.

FIG. 1B depicts a representative assembly pipeline using an mdBG according to a preferred embodiment of this disclosure. The region above the dotted line corresponds to analyses taking place in base space 110, wherein the region below the dotted line corresponds to analyses taking place in minimizer space 112. As will be described in more detail below, the input reads 114 are scanned sequentially, and all l-mers that belong to a pre-selected set of universe minimizers are identified. Each read is then represented as an ordered list of the selected minimizers, and k-min-mers are collected from the minimizer-space representation of reads using a sliding window of length k. These operations are represented by the convert-into-minimizer space operation 116. At step 118, a minimizer-space de Bruijn graph (mdBG) is then constructed from the set of all k-min-mers (defined below) and, at step 120, this graph is simplified in order to reduce ambiguity and remove errors. The resulting mdBG is then converted back into base space at step 212 by concatenating the base-space sequences spanned by the minimizers in the mdBG, and the result—a set of contigs 124—is reported. A contig (from “contiguous”) is a set of overlapping DNA segments that together represent a consensus region of DNA.

FIG. 1C depicts an operation of a minimizer-space partial order alignment (POA) algorithm that provides error correction. In particular, this operation corrects for read errors by performing minimizer-space partial order alignment (POA), in which sequencing errors in a query read are corrected by aligning other reads from the same genomic region to the query in minimizer space. In this example, the partial order alignment (POA) procedure is carried out with a toy dataset of 4 reads. Reference numeral (1) here depicts a set of error-prone reads and their ordered lists of minimizers (l=2), with sequencing errors and the minimizers that are created as a result of errors denoted in colors (insertion as red, deletion as orange, substitution in blue, no errors in green). As depicted at (2), and before minimizer-space error-correction, the ordered lists of minimizers are bucketed using their n-tuples (n=1). At step (3), and for a query ordered list (the first read in the read set), all ordered lists that share an n-tuple with the query are obtained, and the final list of query neighbors are obtained by applying a heuristically determined distance filter dⱼ (e.g., a Jaccard distance threshold of ϕ=0.5). At step (4), a POA graph in minimizer space is constructed by initializing the graph with the query and aligning each ordered list that passed the filter to the graph iteratively (weights of poorly supported edges are shown in red). By taking a consensus path of the graph, and as depicted at step (5), the error in the query is corrected.

The following section provides additional details regarding the above-described techniques.

### Minimizers and De Bruijn Graphs

The variable σ is used as a placeholder for an unspecified alphabet (a non-empty set of characters). Then, Σ_{DNA}={A, C, T, G} is defined as the alphabet containing the four DNA bases. Given an integer l>0, Σ^{l} is the alphabet consisting of all possible strings on Σ_{DNA} of length l. Σ^{l} is an unusual alphabet in the sense that any ‘character’ of Σ^{l} is itself a string of length l over the DNA alphabet.

Given an alphabet σ, a string is a finite ordered list of characters from σ. Note that strings will sometimes be on alphabets where each character cannot be represented by a single alphanumeric symbol. Given a string x over some alphabet σ and some integer n>0, the prefix (respectively the suffix) of x of length n is the string formed by the first (respectively the last) n characters of x.

With the above as background, the following describes the concept of a minimizer as used herein. In particular, consider strings over the alphabet Σ_{DNA} and consider two types of minimizers: universe and window. Further, consider a function ƒ that takes as input a string of length l and outputs a numeric value within range [0, H], where H>0. Usually, ƒ is a 4-bit encoding of DNA, or a random hash function (it does not matter whether the values off are integers or whether H is an integer). Given an integer l>1 and a coefficient 0<δ<1, a universe (1, δ)-minimizer is any string m of length l such that ƒ(m)<δ ·H. Define M_{l,δ} to be the set of all universe (l, δ)-minimizers, and refer to δ as the density of M_{l,δ}. The above definition of a minimizer is in contrast with the classical one; in particular, consider a string x of any length, and a substring (window) y of length w of x. A window l-minimizer of x given window y is a substring m of length l of y that has the smallest value ƒ(m) among all other such substrings in y. Universe minimizers are defined independently of a reference string, unlike window minimizers.

The following is a typical definition of de Bruijn graphs. In particular, and given an alphabet σ and an integer k≥2, a de Bruijn graph of order k is a directed graph where nodes are strings of length k over σ (k-mers), and two nodes x, y are linked by an edge if the suffix of x of length k−1 is equal to the prefix of y of length k−1. This definition also corresponds to a node-centric de Bruijn graph generalized to any alphabet.

### Minimizer-Space De Bruijn Graphs

According to the techniques herein, an algorithm or a data structure operates in minimizer-space when its operations are done on strings over the Σ^{l} alphabet, with characters from M_{l,δ}. Conversely, it operates in base-space when the strings are over the usual DNA alphabet EDNA.

The following introduces the concept of (k, l, δ)-min-mer, or just k-min-mer when clear from the context, defined as an ordered list of k minimizers from M_{l,δ}. This term is used to avoid confusion with k-mers over the DNA alphabet. Indeed, a k-min-mer can be seen as a k-mer over the alphabet Σ^{l}, i.e., a k-mer in minimizer-space. For an integer k>2 and an integer l>1, a minimizer-space de Bruijn graph (mdBG) of order k is defined as de Bruijn graph of order k over the Σ^{l} alphabet. As per the definition in the previous section, and in an mdBG, nodes are k-min-mers, and edges correspond to identical suffix-prefix overlaps of length k−1 between k-min-mers. An example was depicted in FIG. 1A.

The following describes a procedure for constructing mdBGs. First, a set M of minimizers are pre-selected using the universe minimizer scheme from the previous section. Then, reads are scanned sequentially, and positions of elements in M are identified. A multiset V of k-min-mers is created by inserting all tuples of k successive elements in M_{l,δ} found in the reads into a hash table. Each of those tuples is a k-min-mer, i.e., a node of the mdBG. Edges of the mdBG are discovered through an index of all (k−1)-min-mers present in the k-min-mers. As described further below, mdBGs can be simplified and compacted similarly to base-space de Bruijn graphs, using similar rules for removing likely artefactual nodes (tips and bubbles), and by performing path compaction.

By itself the mdBG is insufficient to fully reconstruct a genome in base-space, as in the best case it can only provide a sketch consisting of the ordered list of minimizers present in each chromosome. To reconstruct a genome in base-space, preferably the following operations are used. First, associate to each k-min-mer the substring of a read corresponding to that k-min-mer. The substring likely contains base-space sequencing errors, which are addressed using POA as described below. To deal with overlaps, the positions of the second and second-to-last minimizers in each k-min-mer are tracked. After performing compaction, the base sequence of a compacted mdBG can be reconstructed by concatenating the sequences associated to k-min-mers, making sure to discard overlaps. Note that in the presence of sequencing errors, or when the same k-min-mer corresponds to several locations in the genome, the resulting assembled sequence may be further adjusted using additional base-level polishing.

### Error Correction Using Minimizer-Space Partial Order Alignment (POA)

As previously described (see the discussion regarding FIG. 1C), the input for the minimizer-space POA procedure is a collection of ordered lists of minimizers obtained from all reads in the dataset (one ordered list per read). As seen earlier, the ordered list of minimizers obtained from a read containing sequencing errors will likely differ from that of an error-free read. If the dataset has enough coverage, however, the content of other ordered lists of minimizers in the same genomic region can be used to correct errors in the query read in minimizer-space. To this end, the POA procedure involves first performing a bucketing procedure for all ordered lists of minimizers using each of their n-tuples, where n is a user-specified parameter. After bucketing, and in order to initiate the error-correction of a query, the procedure collects other ordered lists (neighbors) likely corresponding to the same genomic region. A distance metric (e.g., Jaccard or Mash) is then used to pick sufficiently similar neighbors. Once the final set of neighbors that will be used to error-correct the query are obtained, a partial order alignment (POA) procedure is executed, but with the following modifications: (a) a node in the POA graph is a minimizer instead of an individual base, (b) directed edges represent whether two minimizers are adjacent in any of the neighbors, and (c) edge weights represent the multiplicity of the edge in all of the neighbor ordered lists. After constructing the minimizer-space POA by aligning all neighbors to the graph, a consensus (the best-supported traversal through the graph) is generated. Once the consensus is obtained in minimizer-space, the query ordered list of minimizers is replaced with the consensus; this process is then repeated until all reads are error-corrected. To recover the base-space sequence of the obtained consensus after POA, the sequence spanned by each pair of nodes in the edges is stored, and a base-space consensus is generated by concatenating the sequences stored in the edges of the consensus.

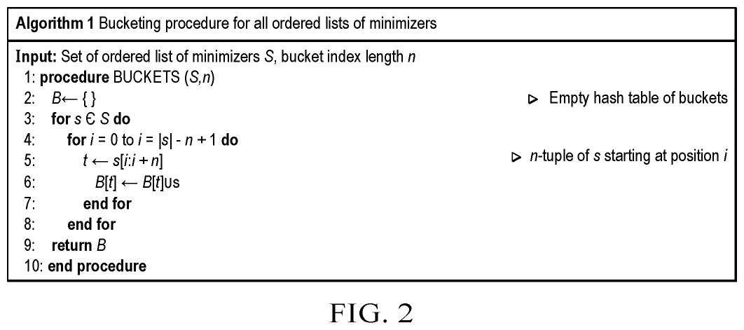

The minimizer-space partial order alignment procedure is depicted in additional detail in FIGS. 2-4. In particular, FIG. 2 (“Algorithm 1”) depicts a POA bucketing and preprocessing routine. In this process, all tuples of length n of an ordered list of minimizers are computed using a sliding window (lines 4-6), and the ordered list of minimizers itself is stored in the buckets labeled by each n-tuple (line 7). In this approach, bucketing is used as a proxy for set similarity, because each pair of reads in the same bucket will have an n-tuple (the label of the bucket) and will be more likely to come from the same genomic region.

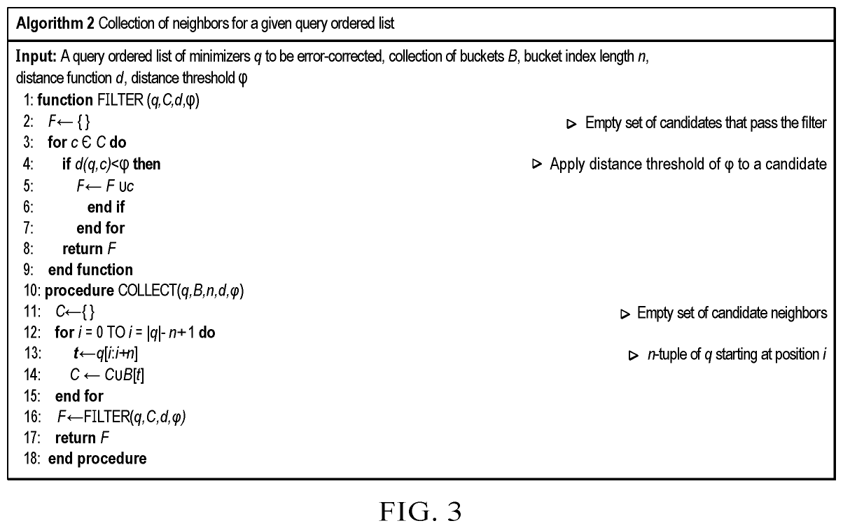

The collection of neighbors for a given query ordered list of minimizers is depicted in FIG. 3 (“Algorithm 2”). This process obtains all n-tuples of a query ordered list, and collects the ordered lists in the previously populated buckets indexed by its n-tuples (lines 10-15). These ordered lists are viable candidates for neighbors because they share a tuple of length at least n with the query ordered list; however, because a query n-tuple may not uniquely identify a genomic region, preferably a similarity filter is applied to further eliminate candidates unrelated to the query. In particular, and using either Jaccard or Mash distance as a similarity metric, and for a user-specified threshold #, the process filters out all candidates that have distance ≥ϕ to the query ordered list to obtain the final set of neighbors used for error-correcting the query (lines 1-9).

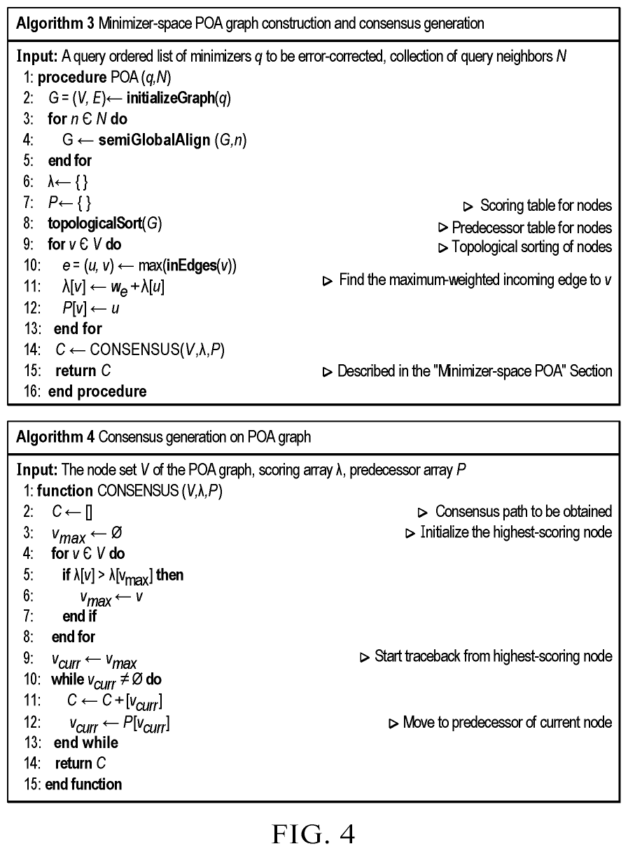

The algorithms for POA graph construction and consensus generation are depicted in FIG. 4. Algorithm 4 here is a canonical POA consensus generation procedure where, as noted above, consensus is being performed in minimizer-space. The minimizer-space POA error-correction procedure is “Algorithm 3,” and it operates as follows. For each neighbor of the query, the process performs semi-global alignment between a neighbor ordered list and the graph, where for two minimizers mᵢ and mⱼ, a match is defined as mᵢ=mⱼ, and a mismatch is defined as mᵢ≠mⱼ (lines 17-19). After building the POA graph G=(V, E) by aligning all neighbors in minimizer space, a consensus is generated to obtain the best-supported traversal through the graph. To this end, the routine first initializes a scoring λ, and sets λ[v]=0 for all v∈V. Then, a topological sort of the nodes in the graph is performed and iterated through the sorted nodes. For each node v, the process then selects the highest-weighted incoming edge e=(u, v) with weight wₑ, and sets λ[v]=wₑ+λ(u). The node u is then marked as a predecessor of v (lines 21-28).

The technique herein provides significant advantages, including the implementation of an ultra-fast minimizer-space de Bruijn graph (mdBG) process geared toward the assembly of long and accurate reads (e.g., such as PacBio HiFi). The solution is fast because it operates in minimizer-space, meaning that the reads, the assembly graph, and the final assembly, are all represented as ordered lists of minimizers, instead of strings of nucleotides. A conversion step then yields a classical base-space representation. Generalizing, the approach herein is used to facilitate genome assembly for state-of-the-art and low-error long-read data. To this end, the approach leverages a minimizer-space de Bruijn graph, which—instead of building an assembly over sequence bases (in a base-space wherein an alphabet sequence comprises nucleotide letters ACGT)—performs assembly in a minimizer-space (wherein an alphabet sequence comprises an ordered sequence of minimizers), and later converts the assembly back to base-space assemblies. Specifically, and in a preferred implementation, each read is initially converted to an ordered sequence of its minimizers. The order of the minimizers is important to maintain, as in this embodiment a goal is to reconstruct the entire genome as an ordered list. To aid in assembly of higher-error rate data, a variant of a partial order alignment (POA) algorithm is implemented. This algorithm, which is designed to operate in minimizer-space instead of base-space, effectively corrects only the bases corresponding to minimizers in the reads. As previously described, the basic approach herein leverages the notion that minimizers can themselves make up atomic tokens of an extended alphabet, which enables efficient long-read assembly that, along with error correction, leads to preserved accuracy. By performing assembly using a minimizer-space de Bruijn graph (in the preferred embodiment), the approach herein reduces the amount of data input to the assembler, preserving accuracy, lowering running time, and decreasing memory usage (e.g., several orders of magnitude) compared to current assemblers.

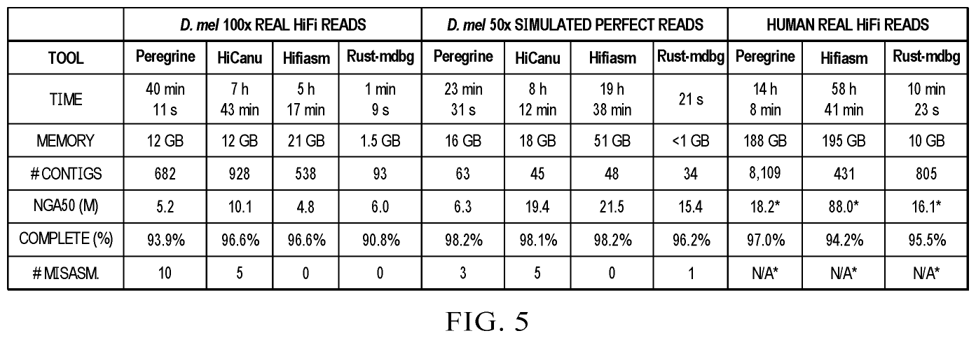

Using the mdBG approach herein to enable long-read DNA genome assembly, orders-of-magnitude improvement in both speed and memory usage over existing methods are achieved, all without compromising accuracy. The approach herein is tantamount to examining a tunable fraction (e.g., only 1%) of the input bases in the data and can be generalized to emerging sequencing technologies. For example, a human genome was assembled in under 10 minutes using 8 processing cores and 10 GB RAM, and 60 Gbp of metagenome reads were assembled in 4 minutes using 1 GB RAM. In addition, and as depicted in FIG. 5, a minimizer-space de Bruijn graph-based representation of 661,405 bacterial genomes, comprising 16 million nodes and 45 million edges was constructed and successfully searched for anti-microbial resistance (AMR) genes in 12 minutes.

### Results

The following describes several experimental results of the above-described methods.

### Fast, Memory-Efficient and Highly-Contiguous Assembly of Real HiFi Reads

The above-described method was implemented in software (rust-mdbg). The software was evaluated against three recent assemblers optimized for low-error rate long reads: Peregrine, HiCanu, and hifiasm. In particular, the code was evaluated on real PacBio HiFi reads from D. melanogaster, at 100× coverage, and HiFi reads for human (HG002) at ˜50× coverage, both taken from the HiCanu publication. Because the method does not resolve both haplotypes in diploid organisms, the evaluation was done by comparing against the primary contigs of HiCanu and hifiasm. In the tests with D. melanogaster, the reference genome consisted of all nuclear chromosomes from the RefSeq accession (GCA 000001215.4). Assembly evaluations were performed using QUAST v5.0.2, and run with parameters recommended in the HiCanu publication. QUAST aligns contigs to a reference genome, allowing computation of contiguity and completeness statistics that are corrected for misassemblies (NGA50 and Genome fraction metrics respectively in Table 1, described below). Assemblies were all run using 8 threads on a Xeon 2.60 GHz CPU. For rust-mdbg assemblies, contigs shorter than 50 Kbp were filtered out. The results do not report the running time of the base-space conversion step and graph simplifications as they are under 15% of the running CPU time and run on a single thread, taking no more memory than the final assembly size, which is also less memory than the mdBG.

The leftmost portion of Table 1 shown in FIG. 5 shows assembly statistics for D. melanogaster HiFi reads. The software rust-mdbg uses—33× less wall-clock time and 8× less RAM than all other assemblers. In terms of assembly quality, all tools yielded high-quality results. HiCanu had 66% higher NGA50 statistics than rust-mdbg, at the cost of making more misassemblies, 385× longer runtime and 8× higher memory usage. The rust-mdbg code reported the lowest Genome fraction statistics, likely due in part to an aggressive tip-clipping graph simplification strategy, also removing true genomic sequences. The rightmost portion of Table 1 shown in FIG. 5 shows assembly statistics for Human HiFi (HG002) reads. In this test, rust-mdbg performed assembly 81× faster with 18× less memory usage than Peregrine, at the cost of a 22% lower contiguity and 1.5% lower completeness. Compared to hifiasm, rust-mdbg performed 338× faster with 19× lower memory, resulting in a less contiguous assembly (NG50 of 16.1 Mbp vs 88.0 Mbp for hifiasm) and 1.3% higher completeness.

Importantly, the initial unsimplified mdBG for the Human assembly only had around 12 million k-min-mers (seen at least twice in the reads, out of 40 million seen in total) and 24 million edges, which should be compared to the 2.2 Gbp length of the (homopolymer-compressed) assembly and the 100 GB total length of input reads in uncompressed FASTA format. This highlights that the mdBG allows very efficient storage and simplification operations over the initial assembly graph in minimizer-space.

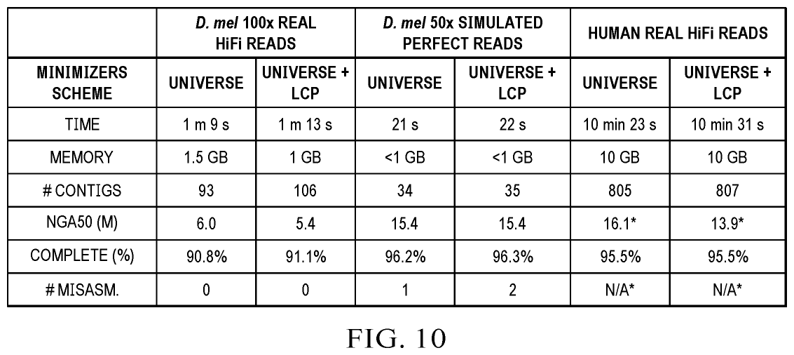

FIG. 10 (Table 3) shows a comparison of assembly statistics between original universe minimizers and universe minimizers with Locally Consistent Parsing (LCP). Locally Consistent Parsing (LCP) describes sets of evenly spaced core substrings of a given length I that cover any string of length n for any alphabet. The set of core substrings can be precomputed such that a string of length n is covered by ˜n/l core substrings on average. Table 3 depicts assembly statistics using both universe minimizers (denoted by “Universe,” same datasets as in Table 1) and universe minimizers with LCP (denoted by “Universe+LCP”) of D. melanogaster real HiFi reads (left), simulated perfect reads (center), and Human real HiFi reads (right), evaluated using the same metrics in Table 1. Parameters for both schemes were k=35, l=12, and δ=0.002 for D. melanogaster, and k=21, l=14, and δ=0.003 for Human.

Minimizer-Space POA Enables Correction of Reads with Higher Sequencing Error Rates

As noted above, the approach herein also leverages minimizer-space partial order alignment (POA) to tackle sequencing errors. To determine the efficacy of minimizer-space POA and the limits of minimizer-space de Bruijn graph assembly with higher read error rates, experiments were performed on a smaller dataset. In particular, reads for a single Drosophila chromosome at various error rates were simulated, and mdBG assembly was performed with and without POA.

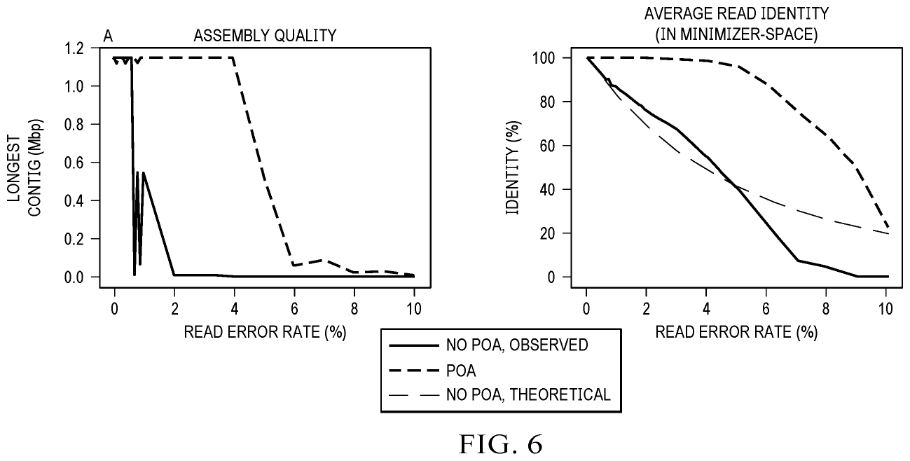

FIG. 6 (left panel) shows that the original implementation without POA is able to reconstruct the complete chromosome into a single contig up to error rates of 1%, after which the chromosome is assembled into ≥2 contigs. With POA, an accurate reconstruction as a single contig is obtained with error rates up to 4%. The results further verified that, up to 3% error rate, the reconstructed contig corresponds structurally exactly to the reference, apart from the base errors in the reads. At 4% error rate, a single uncorrected indel in minimizer-space introduces a ˜1 Kbp artificial insertion in the assembly. FIG. 6 (right panel) indicates that the minimizer-space identity of raw reads linearly decreases with increasing error rate. With POA, near-perfect correction can be achieved up to ˜4% error rate, with a sharp decrease at >5% error rates but still with an improvement in identity over uncorrected reads. With POA, the runtime was around 45 seconds and 0.4 GB of memory, compared to under 1 second and <30 MB of memory without POA.

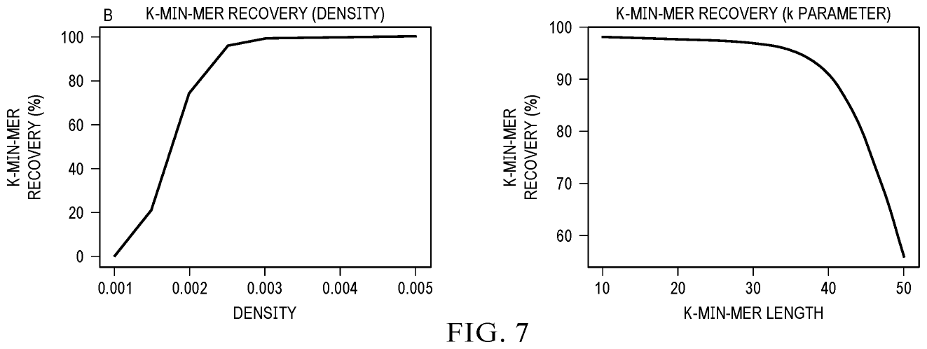

FIG. 7 are graphs depicting the robustness of rust-mdbg assemblies by varying certain parameters (density and k) on whole-genome D. melanogaster simulated perfect reads. The proportion of recovered k-min-mer values is reported in both plots. The left panel shows recovery rates for k=30, 1=12, and varying 6 from 0.001 to 0.005, with good recovery (≥90%) occurring with δ≥0.0025). The right panel shows recovery rates for l=12, δ=0.003, and varying k from 10 to 50, again with good recovery with k≥40.

Pangenome mdBG

The mdBG approach herein was applied to represent a recent collection of 661,405 assembled bacterial genomes. The mdBG construction with parameters k=10, l=12, and δ=0.001 took 3 h50 m wall-clock running time using 8 threads, totaling 8 hours CPU time (largely IO-bound). The memory consumption was 58 GB and the total disk usage was under 150 GB. Increasing δ to 0.01 yields a finer-resolution mdBG but increases the wall-clock running time to 13 h30 m, the memory usage to 481 GB, and the disk usage to 200 GB.

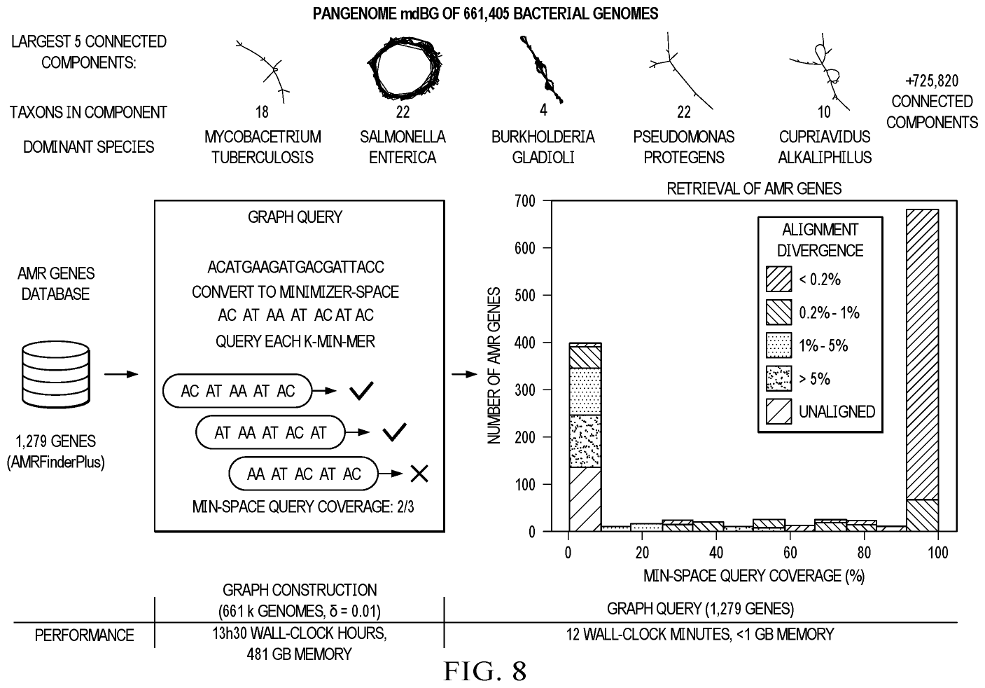

Referring to FIG. 8, a complete 6=0.001 pangenome mdBG was constructed for the whole 661.405 bacterial collection, and the first five connected components are displayed here (using Gephi software) in the top panel. Each node is a k-min-mer, and edges are exact overlaps of k−1 minimizers between k-min-mers. The middle panel depicts a collection of anti-microbial resistance gene targets converted into minimizer space, then each k-min-mer is queried in a 661,405 bacterial pangenome graph (δ=0.01) yielding a bimodal distribution of gene retrieval: genes with high identity (99%+) to those in the pangenome are found, while those with lower identity are not found. The histogram is annotated by the minimal sequence divergence of each gene as aligned by minimap2 to the pangenome over 90% of its length. The bottom panel depicts runtime and memory usage for the δ=0.01 graph construction and query. Note that the graph need only be constructed once in a preprocessing step.

In this experiment, and as expected, several similar species are represented within each connected component. The entire graph consisting of 16 million nodes and 45 million edges (5.3 GB compressed GFA) was much smaller than the original sequences (1.4 TB lz4-compressed).

To illustrate a possible application of this pangenome graph, queries for the presence of AMR genes were performed in the δ=0.01 mdBG; 1,502 targets from the NCBI AMRFinderPlus ‘core’ database (the whole amr_targets.fa file as of May 2021) were retrieved and each gene converted into minimizer-space, using parameters k=10, l=12, δ=0.01. Of these, 1,279 genes were long enough to have at least one k-min-mer (on average 10 k-min-mers per gene). Querying those k-min-mers on the mdBG, on average 61.2% of the k-min-mers per gene were successfully retrieved; however, the retrieval distribution is bimodal: 53% of the genes have ≥99% k-min-mers found, and 31% of the genes have ≤10% k-min-mers found. Further investigation of the genes missing from the mdBG was done by aligning the 661k genomes collection to the genes (in base-space) using minimap2 (7 hours running time over 8 cores). A significant portion of genes (141, 11%) could not be aligned to the collection. Also, k-min-mers of genes with aligned sequence divergence of 1% or more (267, 20%) did not match k-min-mers from the collection, and therefore had zero minimizer-space query coverage. Finally, although sequence queries on a text representation of the pangenome graph were performed, in principle the graph could be indexed in memory to enable instantaneous queries at the expense of higher memory usage.

This experiment illustrates the ability of mdBG to construct pangenomes larger than supported by any other known method, and those pangenomes record biologically useful information such as AMR genes. Long sequences such as genes (containing at least 1 k-min-mer) can be quickly searched using k-min-mers as a proxy. There is nevertheless a trade-off of minimizer-space analysis that is akin to classical k-mer analysis: graph construction and queries are extremely efficient, however, they do not capture sequence similarity below a certain identity threshold (in this experiment, around 99%). Yet, the ability of the mdBG to quickly enumerate which bacterial genomes possess any AMR gene with high similarity provides a potential significant boost to AMR studies.

Highly-Efficient Assembly of Real HiFi Metagenomes Using mdBG

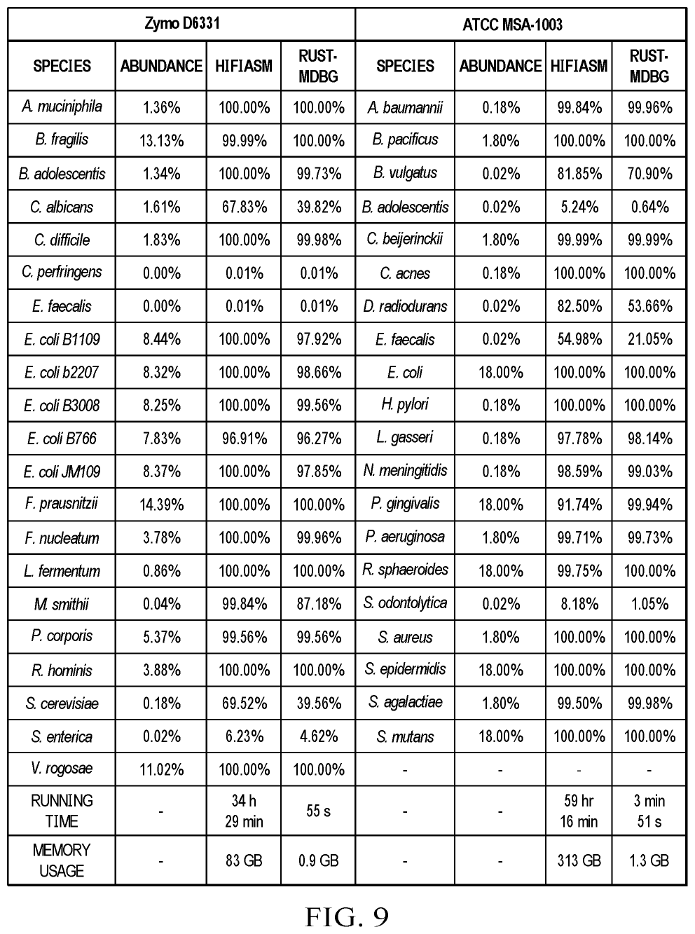

Assembly of two real HiFi metagenome datasets (mock communities Zymo D6331 and ATCC MSA-1003, accessions SRX9569057 and SRX8173258) was also performed. The method was run with the same parameters as in the human genome assembly for the ATCC dataset, and with slightly tuned parameters for the Zymo dataset (see Section “Genome assembly tools, versions and parameters.” Table 2 shown in FIG. 9 shows the results of rust-mdbg assemblies in comparison to hifiasm-meta, a metagenomespecific flavor of hifiasm. The Abundance column shows the relative abundance of the species in the sample. The two rightmost columns show the species completeness of the assemblies as reported by metaQUAST. As this data demonstrates, rust-mdbg achieves roughly two orders of magnitude faster and more memory-efficient assemblies, while retaining similar completeness of the assembled genomes. Although rust-mdbg metagenome assemblies are consistently more fragmented than hifiasm-meta assemblies, the ability of rust-mdbg to very quickly assemble a metagenome enables instant quality control and preliminary exploration of gene content of microbiomes at a fraction of the computing costs of current tools.

### Enabling Technologies

Aspects of this disclosure may be practiced, typically in software, on one or more machines or computing devices. More generally, the techniques described herein are provided using a set of one or more computing-related entities (systems, machines, processes, programs, libraries, functions, or the like) that together facilitate or provide the described functionality described above. In a typical implementation, a representative machine on which the software executes comprises commodity hardware, an operating system, an application runtime environment, and a set of applications or processes and associated data, that provide the functionality of a given system or subsystem. As described, the functionality may be implemented in a standalone machine, or across a distributed set of machines. A computing device connects to the publicly-routable Internet, an intranet, a private network, or any combination thereof, depending on the desired implementation environment.

One implementation may be a machine learning-based computing platform. One or more functions of the computing platform may be implemented in a cloud-based architecture. The platform may comprise co-located hardware and software resources, or resources that are physically, logically, virtually and/or geographically distinct. Communication networks used to communicate to and from the platform services may be packet-based, non-packet based, and secure or non-secure, or some combination thereof.

Each above-described process or process step/operation preferably is implemented in computer software as a set of program instructions executable in one or more processors, as a special-purpose machine.

Representative machines on which the subject matter herein is provided may be hardware processor-based computers running a Linux operating system and one or more applications to carry out the described functionality. One or more of the processes described above are implemented as computer programs, namely, as a set of computer instructions, for performing the functionality described.

While the above describes a particular order of operations performed by certain embodiments of the invention, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

While the disclosed subject matter has been described in the context of a method or process, the subject matter also relates to apparatus for performing the operations herein. This apparatus may be a particular machine that is specially constructed for the required purposes, or it may comprise a computer otherwise selectively activated or reconfigured by a computer program stored in the computer. Such a computer program may be stored in a computer readable storage medium, such as, but is not limited to, any type of disk including an optical disk, a CD-ROM, and a magnetic-optical disk, a read-only memory (ROM), a random access memory (RAM), a magnetic or optical card, or any type of media suitable for storing electronic instructions, and each coupled to a computer system bus.

A given implementation of the computing platform is software that executes on a hardware platform running an operating system such as Linux. A machine implementing the techniques herein comprises a hardware processor, and non-transitory computer memory holding computer program instructions that are executed by the processor to perform the above-described methods.

There is no limitation on the type of computing entity that may implement a function or operation as described herein.

While given components of the system have been described separately, one of ordinary skill will appreciate that some of the functions may be combined or shared in given instructions, program sequences, code portions, and the like. Any application or functionality described herein may be implemented as native code, by providing hooks into another application, by facilitating use of the mechanism as a plug-in, by linking to the mechanism, and the like.

The functionality may be co-located or various parts/components may be separately and run as distinct functions, perhaps in one or more locations (over a distributed network).

Computing entities herein may be independent from one another, or associated with one another. Multiple computing entities may be associated with a single enterprise entity, but are separate and distinct from one another.

### OTHER APPLICATIONS

The technique described herein—wherein minimizers are used and processed in minimizer (as opposed to base) space is useful for applications other than third generation sequencing technologies. Such other applications include sketching, indexing, and clustering large collections of genomic data, computing evolutionary distances between highly similar genomes, estimating sequence abundances in genomic databases, and fast secondary analyses such as mapping, alignment, classification, or structural variation detection.

While the techniques herein are adapted for processing long reads, this is not a limitation. DNA sequencing implementing the techniques herein may be used to determine the sequence of individual genes, larger genetic regions (i.e. clusters of genes), full chromosomes, or entire genomes of any organism.

As used herein, a preferred approach is to utilize minimizers and the minimizer space. This is not intended as limiting, as the approach herein (processing atomic tokens of an extended alphabet in lieu of processing nucleotides in base space) can be applied with respect to tokens generated in some other manner, or by applying some other function to a DNA sequence. For example, the techniques herein may be practiced using a content sensitive partitioning method, such as locally consistent parsing.

What is claimed here follows below:

1. A method for memory-efficient genomic sequence processing, comprising:
scanning a set of input reads, wherein an input read comprising a string of nucleotides;
in lieu of processing the string of nucleotides, generating a memory-efficient representation of the set of input reads by:
identifying a selected set of minimizers;
representing each input read as an ordered list of the selected set of minimizers to generate a minimizer space representation;
collecting k-min-mers from the minimizer space representation of reads using a sliding window of length k;
constructing a directed graph from the set of collected k-min-mers; and
assembling the set of input reads into a minimizer space assembly using the directed graph, the minimizer space assembly being the memory-efficient representation; and
converting the minimizer space assembly into a single genomic sequence.

2. The method as described in claim 1 wherein the directed graph is a de Bruijn graph.

3. The method as described in claim 2 wherein a minimizer is a sequence of nucleotides.

4. The method as described in claim 1 wherein the single genomic sequence is one of: a human genome, a metagenome, and a pangenome.

5. The method as described in claim 1 further including correcting read errors by performing partial order alignment (POA) in the minimizer space.

6. The method as described in claim 5 wherein the POA corrects sequencing errors in a query read by aligning other reads from a similar genomic region to the query in minimizer space.

7. The method as described in claim 1 wherein converting the minimizer space assembly into the single genomic sequence comprises:
storing a sequence spanned by each pair of nodes in edges of the directed graph; and
generating a base-space consensus by concatenating the sequences stored in the edges.

8. A method for efficient genomic sequence processing, comprising:
receiving a set of input reads;
projecting DNA sequences from the set of input reads into ordered lists of minimizers in a minimizer space;
generating a directed graph comprising nodes and edges, wherein in the minimizer space nodes in the directed graph are k-mers over an alphabet of minimizers;
correcting read errors by performing partial order alignment (POA) in the minimizer space; and
assembling the set of input reads into a single genomic sequence using the directed graph.

9. The method as described in claim 8 wherein assembling the set of input reads into a single genomic sequence comprises:
assembling the set of input reads into a minimizer space assembly using the directed graph; and
converting the minimizer space assembly into the single genomic sequence in a base space.

10. The method as described in claim 8 wherein the directed graph is a de Bruijn graph.

11. The method as described in claim 8 wherein the single genomic sequence is one of: a human genome, a metagenome, and a pangenome.

12. An apparatus for DNA sequencing, comprising:
one or more processors;
computer memory holding computer program code executed by the one or more processors for memory-efficient genomic sequence processing, wherein the computer program code is configured to:
scan a set of input reads, wherein an input read comprising a string of nucleotides;
in lieu of processing the string of nucleotides, generate a memory-efficient representation of the set of input reads by:
identify a selected set of minimizers;
represent each input read as an ordered list of the selected set of minimizers to generate a minimizer space representation;
collect k-min-mers from the minimizer space representation of reads using a sliding window of length k;
construct a directed graph from the set of collected k-min-mers; and
assemble the set of input reads into a minimizer space assembly using the directed graph, the minimizer space assembly being the memory-efficient representation; and
convert the minimizer space assembly into a single genomic sequence.

13. The apparatus as described in claim 12 wherein the directed graph is a de Bruijn graph.

14. The apparatus as described in claim 12 wherein the single genomic sequence is one of: a human genome, a metagenome, and a pangenome.

15. A computer program product comprising a non-transitory computer-readable medium for use in a data processing system for efficient genomic sequence processing, the computer program product hold computer program instructions that, when executed by the data processing system:
receive a set of input reads;
project DNA sequences from the set of input reads into ordered lists of minimizers in a minimizer space;
generate a directed graph comprising nodes and edges, wherein in the minimizer space nodes in the directed graph are k-mers over an alphabet of minimizers;
correct read errors by performing partial order alignment (POA) in the minimizer space; and
assemble the set of input reads into a single genomic sequence using the directed graph.

16. The computer program product as described in claim 15 wherein the computer program instructions that assemble the set of input reads include computer program instructions that:
assemble the set of input reads into a minimizer space assembly using the directed graph; and
convert the minimizer space assembly into the single genomic sequence in a base space.

17. The computer program product as described in claim 15 wherein the directed graph is a de Bruijn graph.

18. The computer program product as described in claim 15 wherein the single genomic sequence is one of: a human genome, a metagenome, and a pangenome.
